(19) United States
(12) Patent Application Publication
(10) Pub. No.: US 2010/0011307 A1
(43) Pub. Date: Jan. 14, 2010

(54) USER INTERFACE FOR BREATHING ASSISTANCE SYSTEM

(75) Inventors: Benjamin Desfossez, Cluses (FR); Patrick Michel, Nancy (FR); Hossein Nadjafizadeh, Villers-Les-Nancy (FR)

Correspondence Address: NELLCOR PURITAN BENNETT LLC, ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/168,981

(22) Filed: Jul. 8, 2008

(51) Int. Cl. G06F 3/048; A61M 16/00

(52) U.S. Cl. 715/764; 128/204.18

(57) ABSTRACT

Systems and methods for providing a user interface for a breathing assistance system are disclosed. A user interface may include a plurality of user-activated buttons configured to receive user input and a display communicatively coupled to the plurality of buttons. The display may include a plurality of function indicators, each function indicator associated with a corresponding one of the user-activated buttons and configured to display a different graphical function representations in different situations, the different graphical function representations indicating different functions of the corresponding user-activated button. The display may also include a modification indicator configured to display different graphical modification representations in different situations to indicate whether or not a selected parameter value may be modified using one or more of the user-activated buttons

### TECHNICAL FIELD

The present disclosure relates generally to breathing assistance systems, and particularly to a user interface for a breathing assistance system.

### BACKGROUND

A breathing assistance system typically delivers pressurized breathing gas to a patient via tubing called a “patient interface” or a “breathing circuit.” The breathing gas typically includes air and/or one or more gasses (e.g., oxygen mixed with the air). The breathing gas delivered to the patient from the breathing assistance system may be humidified and/or heated in the breathing circuit before being delivered to the patient. The breathing assistance system typically increases the pressure in the breathing circuit so that the breathing gas is pushed into the lungs for inspiration, and reduces the pressure in the breathing circuit so that gases in the lungs can be expired and vented to the atmosphere. Typically, one or more breathing assistance system parameters may be determined and/or adjusted prior to and/or during operation, e.g., the mode of ventilation (e.g., CMV (controlled mandatory ventilation), SIMV (synchronized intermittent mandatory ventilation), CPAP (constant positive airway pressure), or bi-level CPAP); the patient's tidal volume (the volume of gas inspired with each breath); the respiratory rate (the number of breaths per minute (BPM)); and/or the O₂ concentration, flow rate, airway pressure, and/or minute volume (the volume inspired and expired in one minute) of breathing gas delivered to the patient.

A user interface may permit a user (e.g., a patient or caregiver) to set, control, and/or adjust the various breathing assistance system parameters. For example, a user interface may include a display, buttons, touch screen, and/or other input means allowing a user to input data and/or set breathing assistance parameters. However, due to the operational complexity of some breathing assistance systems, many users have difficulty using traditional user interfaces.

### SUMMARY

In accordance with the teachings of the present disclosure, disadvantages and problems associated with traditional user interfaces used for breathing assistance systems have been substantially reduced or eliminated.

In accordance with one embodiment of the present disclosure, a user interface may include a plurality of user-activated buttons configured to receive user input and a display communicatively coupled to the plurality of buttons. The display may include a plurality of function indicators, each function indicator associated with a corresponding one of the user-activated buttons and configured to display a different graphical function representations in different situations, the different graphical function representations indicating different functions of the corresponding user-activated button. The display may also include a modification indicator configured to display different graphical modification representations in different situations to indicate whether or not a selected parameter value may be modified using one or more of the user-activated buttons.

In accordance with another embodiment of the present disclosure, a breathing assistance system may include a gas delivery system operable to supply breathable gas, a patient interface configured to interface with a patient for delivering breathable gas to the patient, a connection system configured to communicate breathable gas supplied by the gas delivery system to the patient interface for delivery to the patient, a user interface, and a control module communicatively coupled to the user interface and configured to regulate the delivery of breathable gas to the patient based at least on user input received by the user interface. The user interface may include a plurality of user-activated buttons configured to receive user input and a display communicatively coupled to the plurality of buttons. The display may include a plurality of function indicators, each function indicator associated with a corresponding one of the user-activated buttons and configured to display a different graphical function representations in different situations, the different graphical function representations indicating different functions of the corresponding user-activated button. The display may also include a modification indicator configured to display different graphical modification representations in different situations to indicate whether or not a selected parameter value may be modified using one or more of the user-activated buttons.

In accordance with a further embodiment of the present disclosure, a method for displaying user interface functionality information to a user is provided. The method may include providing a plurality of user-activated buttons configured to receive user input. The method may also include displaying a plurality of function indicators, each function indicator associated with a corresponding one of the user-activated buttons and configured to display a different graphical function representations in different situations, the different graphical function representations indicating different functions of the corresponding user-activated button. The method may further include displaying a modification indicator configured to display different graphical modification representations in different situations to indicate whether or not a selected parameter value may be modified using one or more of the user-activated buttons.

Other technical advantages will be apparent to those of ordinary skill in the ail in view of the following specification, claims, and drawings.

### BRIEF DESCRIPTION OF THE DRAWINGS

A more complete understanding of the present embodiments may be acquired by referring to the following description taken in conjunction with the accompanying drawings, in which like reference numbers indicate like features, and wherein:

FIG. 1 illustrates a block diagram of an example breathing assistance system, in accordance with certain embodiments of the disclosure;

FIG. 2 illustrates an example user interface for a breathing assistance system, in accordance with certain embodiments of the disclosure; and

FIGS. 3A-3F illustrate various example user interface screens displayed to a user interface, in accordance with certain embodiments of the present disclosure.

### DETAILED DESCRIPTION

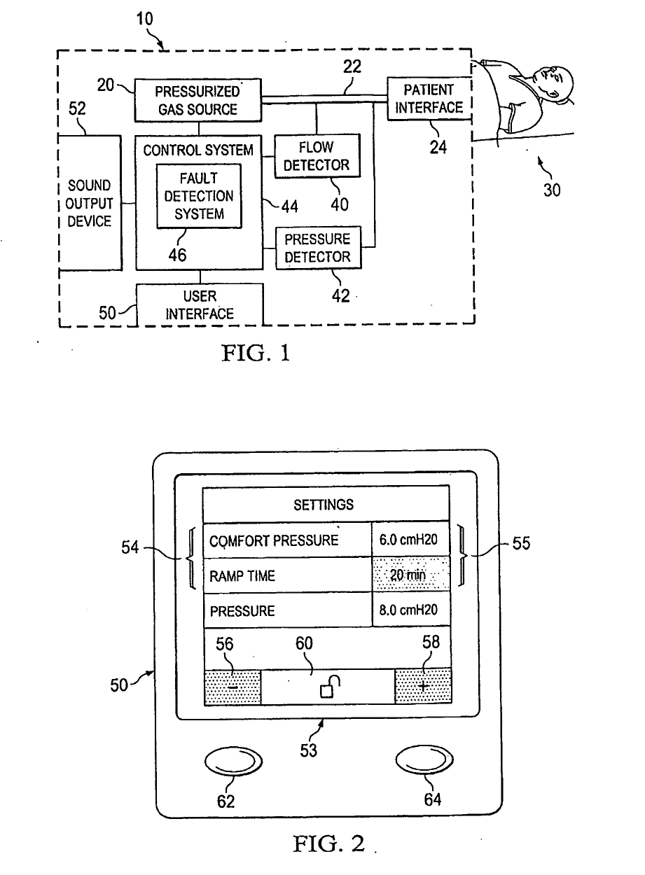

Embodiments of the disclosure may be understood by reference to FIGS. 1 through 3F, wherein like numbers are used to indicate like and corresponding parts.

FIG. 1 illustrates a block diagram of an example breathing assistance system 10, in accordance with certain embodiments of the present disclosure. In general, a user interface may allow a user to view, set, control, and/or adjust parameters associated with the operation of the breathing assistance system.

As used herein, the terms “gas” and/or “breathable gas” may refer to any one or more gases and/or vaporized substances suitable to be delivered to and/or from a patient via one or more breathing orifices (e.g., the nose and/or mouth), such as air, nitrogen, oxygen, any other component of air, CO₂, vaporized water, vaporized medicines, and/or any combination of two or more of the above, for example.

As used herein, the term “patient” may refer to any person or animal that may receive breathing assistance from system 10, regardless of the medical status, official patient status, physical location, or any other characteristic of the person. Thus, for example, patients may include persons under official medical care (e.g., hospital patients), persons not under official medical care, persons receiving care at a medical care facility, persons receiving home care, etc.

As shown in FIG. 1, breathing assistance system 10 may include a gas flow source 20, a connection system 22, a patient interface 24, a flow detector 40, a pressure detector 42, a control system 44, and a user interface 50. Breathing assistance system 10 may comprise any device, apparatus, or system for delivering breathable gas to a patient, e.g., a ventilator, a respirator, a CPAP device, or a BiPAP device. Gas flow source 20 may comprise any system or device suitable for generating and/or delivering pressurized gas (e.g., air and/or oxygen or one or more other supplemental gasses) toward a patient 30, including without limitation, a blower, a compressor, a piston-based device, one or more pressurized gas tanks, one or more gas lines (e.g., from a wall or other source), or any combination thereof.

Connection system 22 may include any system or device suitable for delivering pressurized gas generated by gas flow source 20 towards patient 30, e.g., a connection system and/or other conduits and connection devices. Patient interface 24 may include any system or device suitable for further delivering pressurized gas delivered by connection system 22 to patient 30, e.g., a nasal or face mask, nasal pillows, and/or a tube (e.g., an endotracheal tube, a tracheostomy tube and/or other tracheal tube).

Flow detector 40 may generally be operable to detect the flow rate of gas flowing through one or more conduits of system 10, e.g., the flow rate produced by gas flow source 20 or the flow rate of gas delivered to patient 30. Flow detector 40 may include any number of sensors operable to detect flow rate of a gas and/or any device operable to convert a detected flow rate into electrical signals or otherwise sense flow rate. Flow detector 40 may be placed at any suitable location and in any suitable orientation for sensing flow rate of a gas within breathing assistance system 10. For example, flow detector 40 may be placed within connection system 22, or near gas flow source 20, an air intake port, and/or an air outlet port.

Pressure detector 42 may generally be operable to detect a pressure of gas within one or more conduits of breathing assistance system 10 by gas flow source 20 and/or the pressure of gas delivered to patient 30. Pressure detector 42 may include any number of sensors operable to detect gas pressure and/or any suitable device operable to convert a detected pressure into electrical signals or otherwise sense pressure. Pressure detector 42 may be placed at any suitable location and in any suitable orientation for sensing gas pressure within breathing assistance system 10. For example, pressure detector 42 may be placed within connection system 22, or near gas flow source 20, an air intake port, and/or an air outlet port.

User interface 50 may include any suitable device or devices allowing a user to interface with breathing assistance system 10, e.g., to input desired patient data that may be communicated to control system 44 to control the operation of gas flow source 20 and/or other components of breathing assistance system 10. For example, user interface 50 may allow a user to input (e.g., via buttons, touch screen, and/or other tactile means) one or more of the following patient data: the age, weight, tidal volume capacity, respiratory rate, inhale sensitivity, exhale sensitivity, and/or other characteristics of patient 30, leak settings, rise time, alarm settings, delay, ramp, starting pressure, inhalation:exhalation (I:E) ratio, flow rate, pressure, a selected ventilation program, and/or any other parameter regarding patient 30 and/or the operation of breathing assistance system 10. User interface 50 may also include a display device that may communicate information to a user regarding patient 30 and/or the operation of breathing assistance system device 10.

Control system 44 may generally be operable to process various inputs, e.g., input from user interface 50, ventilation programs stored in memory, and/or feedback from flow detector 40, pressure detector 42, or other variables sensed or otherwise detected by other sensors associated with breathing assistance system 10, and to regulate the operation of gas flow source 20 or other components of breathing assistance system 10 based on such various inputs. Control system 44 may include any suitable system or device for controlling the operation of breathing assistance system 10, including, e.g., a microcontroller, a digital signal processor (DSP), an application specific integrated controller (ASIC), electrically-programmable read-only memory (EPROM), or a field-programmable gate array (FPGA). In some embodiments, control system 44 may include software and/or other executable code for analyzing input signals received from user interface 50 and/or feedback from flow detector 40, pressure detector 42, or other variables sensed or otherwise detected by other sensors associated with breathing assistance system 10 to generate control signals for regulating the operation of breathing assistance system 10. Such software may include any suitable algorithms, logic and/or instructions for processing signals in breathing assistance system 10, and may be stored in any suitable data storage media.

In some embodiments, control system 44 may control the operation of gas flow source 20. For example, where gas flow source 20 comprises a motorized blower, control system 44 may control the operation (e.g., the motor speed and on/off control) of the blower. In addition, control system 44 may generate sound signals to be broadcast by breathing assistance system 10, such as user feedback (e.g., instructions or other words) or other sounds regarding the operation of breathing assistance system 10. For example, control system 44 may monitor the operation of breathing assistance system 10 and, when appropriate, generate alarm signals (e.g., a siren, buzzer, or words) to be broadcast by a sound output device 52. In the same or alternative embodiments, control system 44 may communicate user feedback to user interface 50 for display to a user (e.g., via a display device), and/or receive input from a user via user interface.50, as described in further detail below.

Thus, control system 44 may provide, without limitation, any or all of the following functions: (a) controlling the operation of gas flow source 20, (b) monitoring the operation of ventilator 10, (c) generating user feedback signals to be broadcast by sound output device 52, (d) generating output to be displayed to user interface 50, and/or (e) receiving user input from user interface 50.

Sound output device 52 may generally be operable to output sound signals generated by control system 44, for example, user feedback and/or alarms. Sound output device 52 may include a speaker and an audio driver operable to control the speaker. In some embodiments, sound output device 52 may simultaneously broadcast multiple sound signals.

FIG. 2 illustrates an example user interface 50 for breathing assistance system 10 in accordance with one embodiment of the disclosure. As shown in FIG. 2, user interface 50 may include a display 53 and user input buttons 62 and 64. Display 50 may comprise any system, device, or apparatus configured to display graphic images and/or alphanumeric characters recognizable to a user, and may include, for example, a liquid crystal display (LCD), a cathode ray tube (CRT), a matrix of light emitting diodes (LEDs), or a matrix of organic light emitting diodes (OLEDs). In some embodiments, display 50 may include an illumination device (e.g., in an LCD, a cold cathode florescent lamp or CCFL) or any other suitable device for providing illumination for a display.

Each of buttons 62 and 64 may be any system, device, or apparatus configured to, when pressed or actuated by a user, communicate signals indicative of a user selection to control system 44 and/or another component of breathing assistance system 10. A user's actuation of button 62 and/or 64 may cause a change of an operational parameter of breathing assistance system 10, a change in operation of breathing assistance system 10, and/or a change in the contents of display 53.

Display 53 may include a listing of parameters 54, parameter values 55 associated with parameters 54, function indicators 56 and 58, and modification indicator 60. Listing of parameters 54 may include one or more parameters related to the patient 30 and/or the operation of breathing assistance system 10. For example, parameters 54 may include a mode of breathing assistance (e.g., CMV (controlled mandatory ventilation), SIMV (synchronized intermittent mandatory ventilation), CPAP (constant positive airway pressure), or bi-level CPAP); the tidal volume of patient 30 (the volume of gas inspired with each breath); the respiratory rate of patient 30 (the number of breaths per minute (BPM)); and/or the O₂ concentration, flow rate, airway pressure, and/or minute volume (the volume inspired and expired in one minute) of breathing gas delivered to the patient 30. Parameter values 55 may include operational values (e.g., settings and/or measured values) associated with the displayed parameters 54.

Function indicators 56 and 58 may provide a dynamic graphical indication of the functionality of buttons 62 and 64. For example, in the screen displayed in FIG. 2, function indicator 56 displays a minus sign, which may indicate that pressing button 62 associated with function indicator 56 may reduce a selected parameter value 55 (e.g., the parameter value for the ramp time, as shown in FIG. 2). As another example, in the screen displayed in FIG. 2, function indicator 58 displays a plus sign, which may indicate that pressing button 64 associated with function indicator 58 may increase a selected parameter value 55 (e.g., the parameter value for the ramp time, as shown in FIG. 2).

Modification indicator 60 may provide a dynamic graphical indication of whether a selected parameter value 55 may be modified by a user via user interface 50. For example, in the screen displayed in FIG. 2, modification indicator 60 displays an icon of an unlocked padlock; which may indicate that a user may modify a selected parameter value 55 (e.g., the parameter value for the ramp time, as shown in FIG. 2). In contrast, if modification indicator 60 displays an icon of a locked padlock, it may indicate that a user may not modify a selected parameter value 55.

Although buttons 62 and 64 are shown as separate from display 53, in certain embodiments, display 53 may include a touch screen wherein buttons 62 and 64 are an integral part of display 53. In addition, buttons 62 and 64 and their respective function indicators may be integral to the same element. For example, button 62 and function indicator 56 may be combined, and such combined element may display a graphical representation of the functionality of that element (e.g., the combined element may display a minus sign to indicate that a selected parameter value 55 may be decreased). Similarly, button 64 and function indicator 58 may be combined, and such combined element may display a graphical representation as to the functionality of that element (e.g., the combined element may display a plus sign to indicate that a selected parameter value 55 may be increased).

In addition, although FIG. 2 depicts user interface 50 as including buttons 62 and 64, user interface 50 may include any suitable number of buttons each having any suitable functionality. For example, user interface 50 may include a button allowing a user to confirm a selection (e.g., similar to an “Enter” or “Return” key on a computer keyboard) and/or may include buttons allowing a user to scroll through options and/or parameters on display 53.

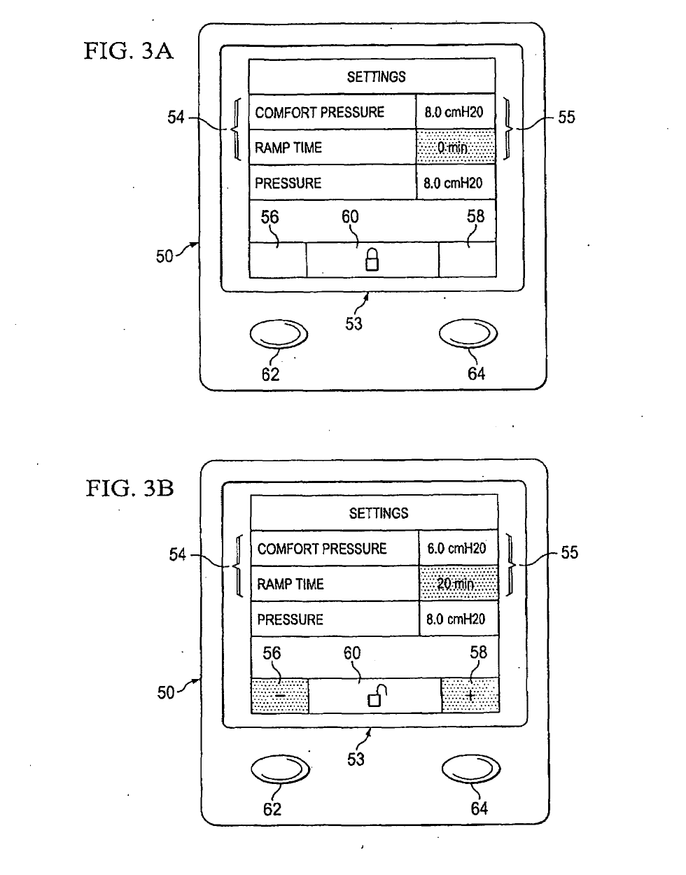

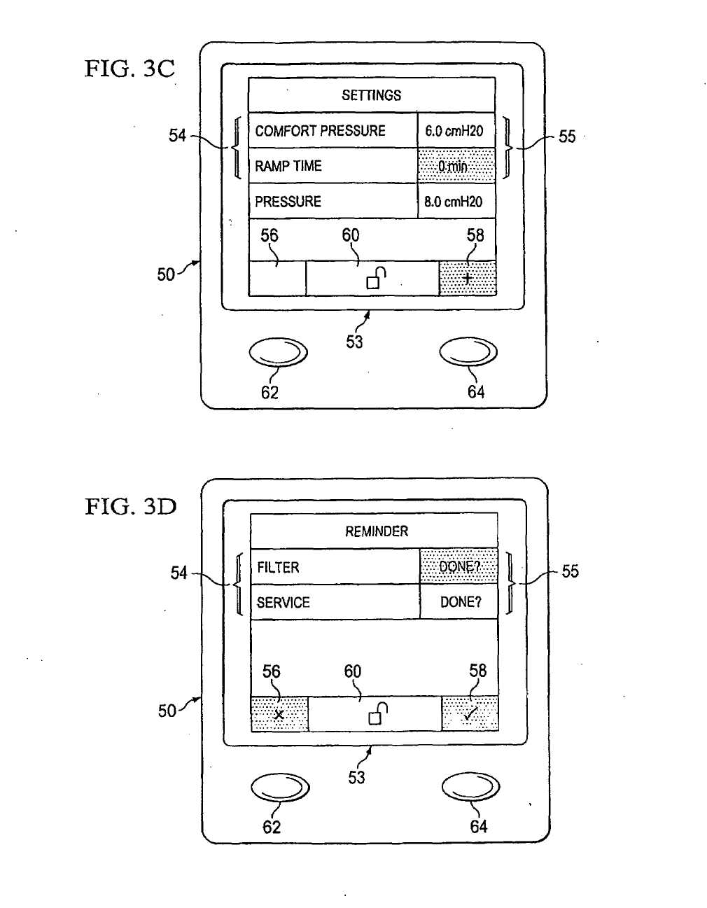

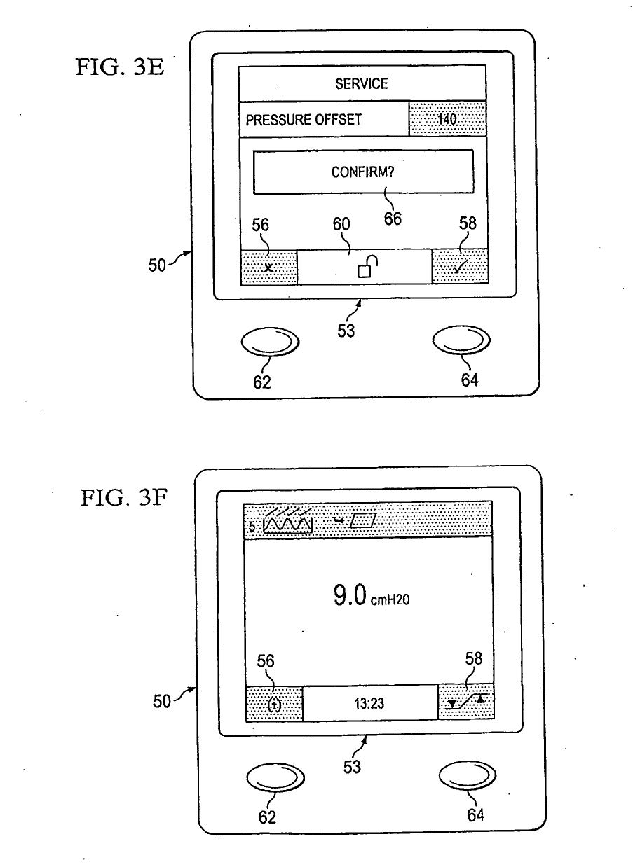

FIGS. 3A-3F illustrate various example user interface screens that may be displayed to display 53 of user interface 50 depending upon the functionality of buttons 62 and 64 and/or the ability to modify a selected parameter value 55, in accordance with the present disclosure. For example, FIG. 3A depicts an example screen that may be displayed at display 53 in the event that a selected parameter value 55 may not be modified by a user. As shown in FIG. 3A, the parameters 54 of “comfort pressure” and “pressure” have each been set to 8.0 cm H₂O. Accordingly, a breathing assistance system 10 providing such pressures may not need to ramp the desired comfort pressure to an operating pressure if such values are identical, and the ramp time between the two pressures is zero. Thus, if a user selects the parameter value 55 associated with the parameter “ramp time” (as indicated by the shading in FIG. 3A), function indicators 56 and 58 may indicate that associated buttons 62 and 64 are not functional to modify the ramp time parameter (e.g., by appealing blank as shown in FIG. 3A; by a “grayed out” icon; or using an icon indicating the parameter may not be modified, such as an “x,” for example). In addition, modification indicator 60 may indicate that the selected parameter “ramp time” may not be modified (e.g., modification indicator 60 may display an icon of a locked padlock as shown in FIG. 3A, or display appropriate text such as “locked,” for example).

As another example, FIG. 3B depicts an example screen that may be displayed to display 53 in the event that a selected parameter value 55 may be modified by a user with each button 62 and 64 having functionality to modify such parameter value. As shown in FIG. 3B, the parameters 54 of “comfort pressure” and “pressure” have been set to 6.0 cm H₂O and 8.0 cm H₂O, respectively. Accordingly, a breathing assistance system 10 providing such pressures may ramp from the desired comfort pressure to an operating pressure according to a user setting. Thus, if a user selects the parameter value 55 associated with the parameter “ramp time,” function indicators 56 and 58 may indicate that associated buttons 62 and 64 are functional to modify the ramp time parameter. For example, function indicator 56 may display a minus sign indicating that button 62 may decrease the ramp time and/or function indicator 58 may display a plus sign indicating that button 64 may increase the ramp time as shown in FIG. 3B. In addition, modification indicator 60 may indicate that the selected parameter “ramp time” may be modified (e.g., modification indicator 60 may display an icon of an unlocked padlock as shown in FIG. 3B; or display appropriate text such as “unlocked,” for example).

As another example, FIG. 3C depicts an example screen that may be displayed to display 53 in the event that a selected parameter value 55 may be modified by a user, with only one of buttons 62 and 64 having functionality to modify such parameter value. As shown in FIG. 3C, the parameters 54 of “comfort pressure” and “pressure” have been set to 6.0 cm H₂O and 8.0 cm H₂O, respectively. Accordingly, a breathing assistance system 10 providing such pressures may ramp from the desired comfort pressure to an operating pressure according to a user setting. However, if the selected parameter value 55 for ramp time is at a minimum or maximum, one of buttons 62 and 64 may be functional to modify the selected parameter value 55, while the other button 62 and 64 may not. Thus, if a user selects the parameter value 55 associated with the parameter “ramp time” and the parameter value 55 is at its minimum value, function indicator 56 may indicate that associated button 62 is not functional to modify the ramp time parameter and/or function indicator 58 may indicate that associated button 64 is functional to modify the ramp time parameter. For example, function indicator 56 may appear blank to indicate that button 62 is not functional to modify the ramp time and/or function indicator 58 may display a plus sign indicating that button 64 may increase the ramp time as shown in FIG. 3C. Alternatively, function indicator 56 may appear as a “grayed out” icon or an icon indicating that the parameter may not be modified, such as an “x” for example. In addition or alternatively, function indicator 58 may appear as an icon indicated that the parameter may be modified, such as a checkmark, for example. In addition, modification indicator 60 may indicate that the selected parameter “ramp time” may be modified (e.g., modification indicator 60 may display an icon of an unlocked padlock as shown in FIG. 3C, or display appropriate text such as “unlocked,” for example).

As a further example, FIG. 3D depicts an example screen that may be displayed to display 53 in the event a selected parameter value 55 may be modified by a user and buttons 62 and 64 having functionality to select such parameter value. As shown in FIG. 3D, a parameter value 55 associated with a filter change is selected. The filter change parameter may have one of two values (e.g., “no” or “false” if the filter has not been changed, “yes” or “true” if the filter has been changed). Thus, if a user selects the filter change parameter value 55, function indicators 56 and 58 may indicate that their associated buttons 62 and 64 may be functional to select a value for such parameter value 55. For example, function indicator 56 may display an “x” to indicate that button 62 is functional to select “no” or “false,” and/or function indicator 58 may display a checkmark to indicate that button 64 is functional to select “yes” or “true” as shown in FIG. 3D. In addition, modification indicator 60 may indicate that the selected parameter value may be modified and/or selected (e.g., modification indicator 60 may display an icon of an unlocked padlock as shown in FIG. 3D, or display appropriate text such as “unlocked,” for example).

As yet another example, FIG. 3E depicts an example screen that may be displayed to display 53 in the event a user is prompted to input a value using button 62 and/or button 64. As shown in FIG. 3E, display 53 has displayed “CONFIRM?” to prompt a user to confirm a particular setting and/or parameter. Accordingly, the user's response to the prompt may have one of two values (e.g., “no” or “false” if the user desires not to confirm, or “yes” or “true” if the user desires to confirm). Thus, when such a prompt is displayed, function indicators 56 and 58 may indicate that their associated buttons 62 and/or 64 may be functional to select a response to the prompt. For example, function indicator 56 may display an “X” to indicate that button 62 is functional to select “no” or “false” and/or function indicator 58 may display a checkmark to indicate that button 64 is functional to select “yes” or “true” as shown in FIG. 3E. In addition, modification indicator 60 may indicate that the displayed prompt may be responded to (e.g., modification indicator 60 may display an icon of an unlocked padlock as shown in FIG. 3E, or display appropriate text such as “unlocked,” for example).

As yet another example, FIG. 3F depicts an example screen that may be displayed to display 53 during operation of breathing assistance system 10. During such operation, a user may press buttons 62 and/or 64 in order to access information menus regarding breathing assistance system 10 and/or access parameter menus allowing the user to modify operational parameters of breathing assistance system 10. Accordingly, function indicators 56 and 58 may display a graphic indicating the functionality of associated buttons 62 and 64. For example, function indicator 56 may display a graphic to indicate that button 62 is functional to access an information menu, and/or function indicator 58 may display a graphic to indicate that button 64 is functional to access a parameter menu as shown in FIG. 3F.

Using the methods and systems disclosed herein, problems associated conventional approaches to breathing assistance system user interfaces may be reduced or eliminated. For example, because the methods and systems disclosed may allow for the functionality of user input buttons to change depending on the context of a display, allow for an indication of such functionality, and allow for an indication of whether parameters displayed may be modified, a user interface may be designed with fewer input buttons (allowing for a compact, space-saving design) while providing an intuitive user experience.

Although the disclosed embodiments have been described in detail, it should be understood that various changes, substitutions and alterations can be made to the embodiments without departing from their spirit and scope.

What is claimed is:

1. A user interface comprising:
a plurality of user-activated buttons configured to receive user input; and
a display communicatively coupled to the plurality of buttons and including:
a plurality of function indicators, each function indicator associated with a corresponding one of the user-activated buttons and configured to display a different graphical function representations in different situations, the different graphical function representations indicating different functions of the corresponding user-activated button; and
a modification indicator configured to display different graphical modification representations in different situations to indicate whether or not a selected parameter value may be modified using one or more of the user-activated buttons.

2. A user interface according to claim 1, wherein one of the graphical function representations indicates that the user-activated button associated with the function indicator is functional to increase the selected parameter value.

3. A user interface according to claim 1, wherein one of the graphical function representations indicates that the button associated with the function indicator is functional to decrease the selected parameter value.

4. A user interface according to claim 1, wherein one of the graphical function representations indicates that the button associated with the function indicator is not functional.

5. A user interface according to claim 1, wherein one of the graphical function representations indicates that the button associated with the function indicator is functional to select the parameter value.

6. A user interface according to claim 1, wherein one of the graphical function representations indicates that the button associated with the function indicator is functional to respond to a prompt displayed on the display.

7. A user interface according to claim 1, wherein one of the graphical function representations indicates that the button associated with the function indicator is functional to access a menu.

8. A breathing assistance system comprising:
a gas delivery system operable to supply breathable gas;
a patient interface configured to interface with a patient for delivering breathable gas to the patient;
a connection system configured to communicate breathable gas supplied by the gas delivery system to the patient interface for delivery to the patient;
a user interface including:
a plurality of user-activated buttons configured to receive user input; and
a display communicatively coupled to the plurality of buttons and including:
a plurality of function indicators, each function indicator associated with a corresponding one of the user-activated buttons and configured to display a different graphical function representations in different situations, the different graphical function representations indicating different functions of the corresponding user-activated button; and
a modification indicator configured to display different graphical modification representations in different situations to indicate whether or not a selected parameter value may be modified using one or more of the user-activated buttons; and
a control module communicatively coupled to the user interface and configured to regulate the delivery of breathable gas to the patient based at least on user input received by the user interface.

9. A breathing assistance system according to claim 8, wherein one of the graphical function representations indicates that the user-activated button associated with the function indicator is functional to increase the selected parameter value.

10. A breathing assistance system according to claim 8, wherein one of the graphical function representations indicates that the button associated with the function indicator is functional to decrease the selected parameter value.

11. A breathing assistance system according to claim 8, wherein one of the graphical function representations indicates that the button associated with the function indicator is not functional.

12. A breathing assistance system according to claim 8, wherein one of the graphical function representations indicates that the button associated with the function indicator is functional to select the parameter value.

13. A breathing assistance system according to claim 8, wherein one of the graphical function representations indicates that the button associated with the function indicator is functional to respond to a prompt displayed on the display.

14. A breathing assistance system according to claim 8, wherein one of the graphical function representations indicates that the button associated with the function indicator is functional to access a menu.

15. A method for displaying user interface functionality information to a user, including:
providing a plurality of user-activated buttons configured to receive user input;
displaying a plurality of function indicators, each function indicator associated with a corresponding one of the user-activated buttons and configured to display a different graphical function representations in different situations, the different graphical function representations indicating different functions of the corresponding user-activated button; and
displaying a modification indicator configured to display different graphical modification representations in different situations to indicate whether or not a selected parameter value may be modified using one or more of the user-activated buttons.

16. A method according to claim 15, wherein one of the graphical function representations indicates that the user-activated button associated with the function indicator is functional to increase the selected parameter value.

17. A method according to claim 15, wherein one of the graphical function representations indicates that the button associated with the function indicator is functional to decrease the selected parameter value.

18. A method according to claim 15, wherein one of the graphical function representations indicates that the button associated with the function indicator is not functional.

19. A method according to claim 15, wherein one of the graphical function representations indicates that the button associated with the function indicator is functional to select the parameter value.

20. A method according to claim 15, wherein one of the graphical function representations indicates that the button associated with the function indicator is functional to respond to a prompt displayed on the display.

21. A method according to claim 15, wherein one of the graphical function representations indicates that the button associated with the function indicator is functional to access a menu.
